(19) United States
(12) Patent Application Publication
(10) Pub. No.: US 2026/0118255 A1
(43) Pub. Date: Apr. 30, 2026

(54) SYSTEMS AND METHODS FOR USING HYPERSPECTRAL FEATURES FOR MINERALOGICAL INFORMATION AT WELL SITES

(71) Applicant: Predictive Drill Quality Limited, Sherwood Park (CA)

(72) Inventors: Benoit Rivard, Sherwood Park (CA); Charles Wrightson, Chestermere (CA); Jilu Feng, Edmonton (CA); Tanner Litwin, Calgary (CA)

(73) Assignee: Predictive Drill Quality Limited, Sherwood Park (CA)

(21) Appl. No.: 19/003,543

(22) Filed: Dec. 27, 2024

Related U.S. Application Data
(60) Provisional application No. 63/615,889, filed on Dec. 29, 2023.

(51) Int. Cl. G01N 21/27; E21B 44/00; E21B 49/00; G01N 33/24; G06T 11/20

(52) U.S. Cl. CPC G01N 21/27; E21B 49/005; G01N 33/24; G06T 11/26

(57) ABSTRACT

This disclosure is directed to systems and methods of controlling a drill for an oil or gas well. The method includes extracting drill cuttings from the drill and obtaining a reflectance spectra of the drill cuttings. The reflectance spectra may be communicated over one or more communication networks to a remote repository. The method may further include processing the reflectance spectra at a remote server in communication with the remote repository, wherein processing the reflectance spectra comprises applying a model to obtain values for each of one or more properties of the drill cuttings. The results may be communicated to a receiver and the drilling adjusted.

### CROSS-REFERENCE TO RELATED APPLICATIONS

This application claims priority to U.S. Provisional Application No. 63/615,889, filed Dec. 29, 2023, which is herein incorporated by reference in its entirety.

### FIELD

This disclosure generally relates to the use of hyperspectral features for determining mineralogical information. In more particularity, it relates to using hyperspectral information from samples obtained from an oil or gas well to determine information on the reservoir, and drilling process.

### BACKGROUND

While drilling an oil or gas well, drill cuttings may be obtained reflecting the material through which the drill bit is drilling. Knowing where the drill bit is drilling is helpful to compare the actual formation, with the predicated characteristics. The drill cuttings may also reflect the contents of the reservoir.

Adjustments to the drilling processed based on a drilling plan and information on the location of the drill bit are made while drilling.

Making a timely and accurate assessment of the drilling cuttings is desirable to improving the drilling process and for post drilling completion design.

### SUMMARY

This disclosure is directed to systems and methods of controlling a drill for an oil or gas well. The method includes extracting drill cuttings from the drill and obtaining a reflectance spectra of the drill cuttings. The reflectance spectra may be communicated over one or more communication networks to a remote repository. The method may further include processing the reflectance spectra at a remote server in communication with the remote repository, wherein processing the reflectance spectra comprises applying a model to obtain values for each of one or more properties of the drill cuttings. The results may be communicated to a receiver quickly so that the drilling adjusted.

### BRIEF DESCRIPTION OF THE DRAWINGS

In drawings which illustrate by way of example only a preferred embodiment of the disclosure,

FIG. 1 is a representation of typical phases of a exploitation of an oil or gas reserve.

FIGS. 2A and 2B are representations of a sample spectral chart.

FIG. 3 is a schematic representation of an architecture of an embodiment.

FIG. 4 is a representation of oil production for two wells.

FIG. 5 is a representation of depth properties of a vertical well.

FIG. 6 is a comparison of carbonate mineralogy for three horizontal wells derived from spectra of cuttings.

FIG. 7 shows three rows representing data at corresponding depths for a well.

FIG. 8 represents data relating hardness hyperspec derived from hyperspectral measurements done in accordance with an embodiment, with clay content measurements derived from X-ray fluorescence (typically done post-well completion) collected from lateral cuttings of a sample well.

### DETAILED DESCRIPTION

Exploitation of oil and gas resources typically involve a planning and an execution phase.

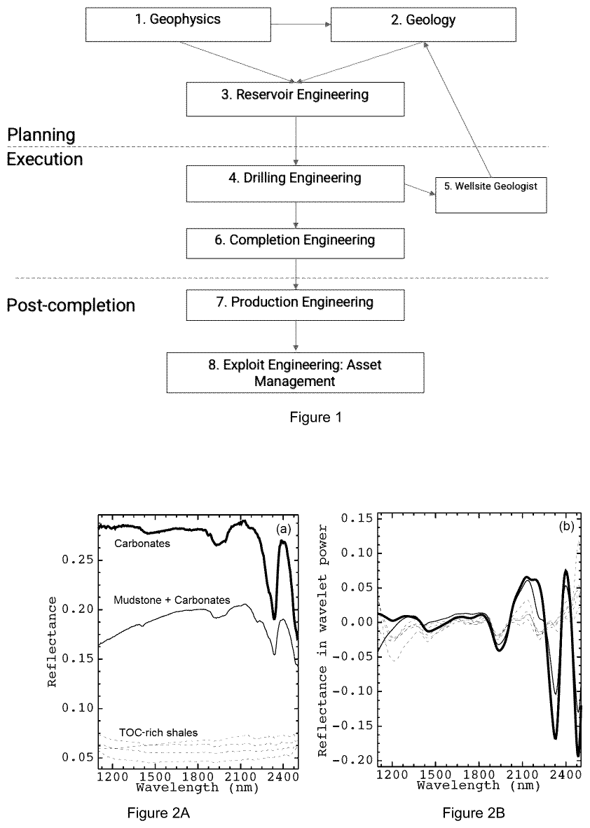

In the planning phase and in reference to FIG. 1, geophysicists (1) may help define and map the play. They provide a very rough definition of the depth, location and areal extent of a play typically by using the results of seismic surveys.

Geologists (2) may help define and map the play using historical well logs and cores that have been cut from legacy wells in the area of interest. Core and logs may be used to assess important reservoir parameters. Parameters may include porosity, permeability, and fluid saturations. These parameters may be used to create play maps that are incorporated into hydrocarbon reserves evaluations and are used to pick drilling locations.

Reservoir engineers (3) may incorporate the information provided by the geologists and geophysicists into their estimates of flow behavior within the reservoir. Their objectives among others are to determine drilling locations, forecast production volumes, perform reserve estimates, and develop strategies to maximize hydrocarbon recovery. Parameters that are typically of importance for a reservoir engineer are permeability, porosity, pore structure & fluid saturation.

In the execution phase, actual wells are drilled into the reservoir. Drilling engineers (4) typically incorporate information provided by geologists, reservoir engineers and completions engineers into their drilling designs. They may select the preferred landing depth, plan the drilling program, and monitor the drilling process to ensure the drilling budget is not exceeded. A typical objective is to develop strategies to optimize the rate at which they drill the well. They may troubleshoot issues such as a drop-in rate of penetration and loss of circulation.

Wellsite geologists (5) may interpret drill cuttings to track where the drill bit is in formation. Drill cuttings are generally bits of solid material brought up from drill bit in the drilling mud. Drill cuttings may be separated from the drilling mud, including to allow for analysis of the drill cuttings, obtaining samples and for disposal.

The wellsite geologists may create reports that summarize various sections of the wellbore based on the drill cuttings and other information, and these reports may be used by the office geologists and reservoir engineers to characterize sections of the wellbore.

Completion engineers (6) may incorporate the information provided by the geologists and reservoir engineers into their completion designs. They may select the placement and design of each stage in the hydraulic fracturing operation. Considerations among others are hardness, the presence of barriers and baffles, stress regimes, fracture behavior.

Post completion, production engineers (7) may incorporate the information provided by the geologists and reservoir engineers into their lifting equipment designs with the goal of achieving the optimal equipment size, or lifting capacity, relative to the deliverability of the reservoir. Considerations may include reservoir permeability, fluid saturations, porosity and pressure.

Exploit engineers (8) may incorporate the information provided by geologists, geophysicists, reservoir engineers, drilling engineers, completions engineers and production engineers to maximize the net present value (NPV) of the asset.

Infrared reflectance spectra from rock material obtained from oil and gas reservoirs may be related to several reservoir rock properties including the total organic carbon (see for example, Rivard, B., Harris, N., Feng, J., and Dong, T. 2018. Inferring TOC and major element geochemistry and mineralogical characteristics of shale core from hyperspectral imagery. AAPG Bull. 102(10): 2101-2121, incorporated by reference), mineralogy, and rock hardness (see for example, Harris, N., Rivard, B., Feng, J., and Moghadam, A. 2019. Obtaining geomechanical information from hyperspectral imaging of a shale core, Horn River Basin, western Canada. AAPG annual convention, San Antonio, May 19-22, 2019, incorporated by reference). These properties may affect drilling and reservoir performance.

Reflectance spectra may be collected using an instrument such as a point or imaging spectrometer operating in the visible region, short-wave infrared, mid-wave infrared and long-wave infrared. The operating range may preferably include spectrum 0.4-22 mm. Typically, the spectrometer has an internal light source to illuminate the sample. The reflectance spectrum is obtained from the ratio of each sample measurement to that of a standard surface of known reflectance illuminated with the same geometry.

A spectrum is typically taken within a few seconds of contacting the instrument with rock material. Features that may be revealed in the spectra may include, but are not limited to, absorption features, reflectance features, peaks, slopes, ratios between spectral bands, and wavelet spectrograms.

The spectrum may be represented as a ratio of reflectance to a standard surface of known reflectance. The spectrum may also be represented in wavelet power. With reference to FIGS. 2A and 2B, the reflectance of several materials may be represented over a range of wavelengths. In the figures, reflectance is graphed of carbonates, a mix of mudstone and carbonates, and several TOC-rich shales. TOC-rich refers to shale with a high Total Organic Carbon. The reflectance may be represented, as in FIG. 2A as a ratio of reflected light from a sample to that of a standard surface. With reference to FIG. 2B, the reflectance may be represented as a wavelet power as determined using continuous wavelet analysis (CWT).

In an embodiment, a sample spectrum may be taken or recorded using the portable spectrometer of drill cuttings. While reference is made to drill cuttings, measurements may be taken of other material obtained from underground such as rock chips, or cores collected at the oil or gas well during drilling. While a portable spectrometer is described in an embodiment, alternatively, an infrared spectrometer(s) and or several spectrometers may be used, such as with sensitive to different wavelengths, could be used to generate info and ensuing results.

In a preferred embodiment, once available the cuttings may be deposited on a surface. Preferably, the cuttings are distributed to cover an area of approximately 1 inch in diameter and several millimeters thick. The instrument measurement window of the spectrometer may be brought into contact with the cuttings and the data collection is triggered during which the sample is illuminated for a few seconds. The measurement window of the spectrometer may be on the order of a centimeter in diameter. In another embodiment, the spectrometer may measure light from a fibre optic bundle with a conical field of view measured in degrees. In such an embodiment, the sample may fill the field of view for collecting a spectrum but may not need to be in contact with the spectrometer.

The spectrum acquired is then sent to the remote data repository electrically as will be described below. The results may be processed, and results returned to the well site, such as by being presented on a web page. This process may be repeated on additional samples, such as when additional samples are extracted from the borehole. The sampling may be done proximate to the borehole after the drill cuttings are separated from the drilling mud.

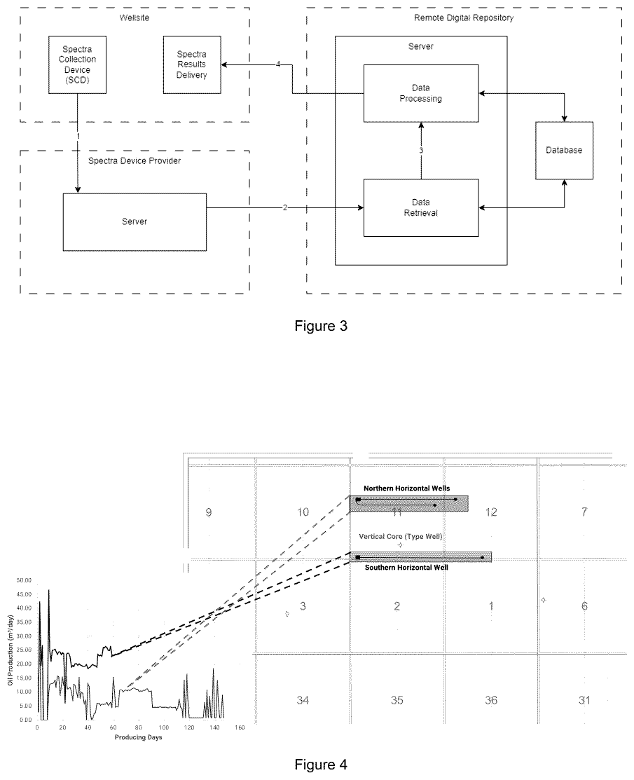

With reference to FIG. 3, in an embodiment, the spectral data captured by the spectrometer is communicated to a remote digital repository. The spectrometer may initiate the transfer simultaneously with the capture of the data or at some time after the data is captured. The transfer may be automatic or initiated by an operator. In an embodiment, the remote digital repository may do preliminary processing of the data and/or maintain an archive of the data.

The remote digital repository may be established by the provider of the spectrometer. The remote digital repository may be one or more servers or part of a cloud-based system, such as provided by Amazon's AWS. The remote digital repository may be managed by the manufacturer of the spectrometer. The communication between the spectrometer and the remote digital repository may use one or more protocols such as HTTP or a proprietary protocol.

The spectral data from spectrometer may be further communicated to a further repository. The further repository may be a further one or more servers or part of a cloud-based system. The remote digital repository may initiate the transfer once it receives the spectral data. The remote digital repository may provide a notification to the further repository that new spectral data is available and the further repository makes a request for the spectral data to initiate a transfer of the spectral data to the further repository.

In some embodiments, the spectral data may be transferred from the spectrometer to the further repository without utilizing the remote digital repository or the spectral data may be transferred to a local computer, and then the data is transferred to the remote digital repository. In other embodiments, some spectral data may be transferred to the further repository directly and some may transfer first to the remote digital repository. For example, spectral data from a first spectrometer may be transferred to the remote digital repository, while spectral data from a second spectrometer may be transferred directly to the further repository.

The remote digital repository may include webhooks to facilitate the transfer of the spectral data to the further repository. The webhook may be a real-time data push system that uses the HTTP protocol. When spectral data is received at the remote digital repository, the data may be further transferred to the further repository. This further transfer may happen immediately or within a short period of time.

The spectral data received at the remote digital repository may be stored for future access, such as in a database. The spectral data at the further repository may be further processed. A queue may be maintained for spectral data for processing with the spectral data processed in the order it is received or some other sequence, such as based on the priority of the spectral data or the job where the spectral data was obtained.

Once processed, as described below, the processed spectral data may be stored and/or a report generated. The analysis may be integrated with the processing of the data in the database and report generation.

The generated report may be communicated to the location where the spectral analysis was captured at the well site, or a further location where engineers associated with the well site are situated. The results may be a time series that is updated in real-time as samples are collected and are available to the wellsite operators to assist with real time decision making. The report may be communicated in a suitable format such as XML, txt, PDF or HTML. For example, with reference to FIG. 6, a graph may be generated with the spectral analysis, that includes each sample and which is updated as new samples are captured at a given well.

Preferably the results are provided to devices at the well site quickly, preferably in less than one minute. The results may assist with drilling decisions and control, such as assessing whether the drill is still in play in the case of a horizonal well, to avoid and minimize high clay horizons, and attempt to drill out of them faster. The results may be incorporated into the drilling operations automatically or provided to operators. Embodiments may also be used for other types of wells and continuous drilling operations where drilling processes can be guided by measurements of rock material by a spectrometer.

Embodiments can yield a real-time or near real-time estimate of several rock characteristics, primarily mineralogical in nature, relevant to drilling decisions that otherwise are not readily available. Real time information can be used for improved reservoir targeting to increase hydrocarbon production and for reducing drilling time which in turn saves costs and reduces environmental impact by lowering water and energy consumption. If the results are provided in approximately 1 minute or less, the users at the wellsite can make decisions based on the results, including based on a depth profile. Access to live clay estimation also serves post drill frac completion design.

If drilling cuttings or rock chips have to be delivered to a lab for analysis, it may take hours or days for the results to be known and drilling decisions to be made. Similarly, if measurement data must be shipped offsite for an analysis, the results may not be readily available for drilling decisions.

Real time mineralogy and hydrocarbon abundance results may not be available with prior technologies, which typically include lengthy delay to access information that precludes the use of such information for real-time drilling decisions. Real time information can be used for improved reservoir targeting to increase hydrocarbon production and for reducing drilling time which in turn saves costs and reduces environmental impact by lowering water and energy consumption.

With reference to FIG. 4, example oil production data is shown over time for two well locations, two northern horizontal wells, and a southern horizontal well. As indicated the oil production per day is indicated with the production for the southern horizontal well being substantially higher than that of the northern wells. The southern well has a much better oil production and sustained production while the northern wells have a lower initial production that also rapidly declines. In the background of FIG. 4, a representation of the physical location of the wells is indicated with reference to a grid coordinates. A red symbol indicates the location of a vertical well.

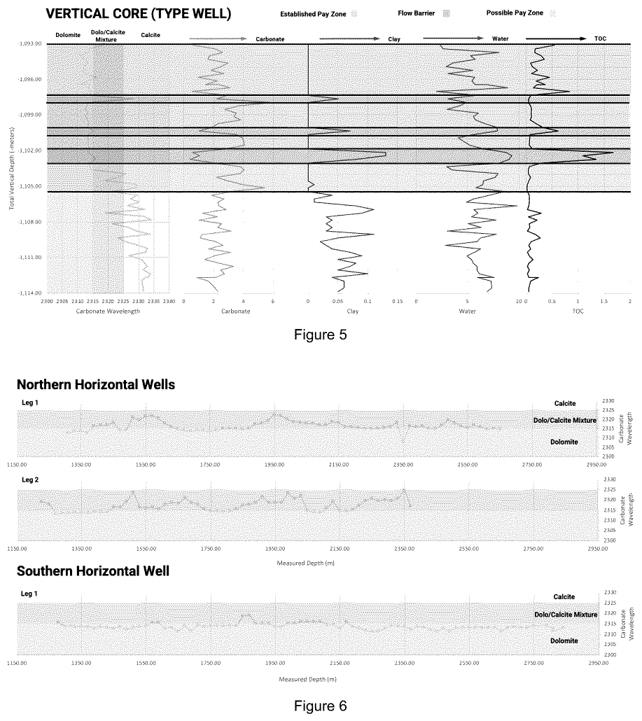

With reference to FIG. 5, depth profiles based on spectral data is indicated for five properties at various depths near a depth of interest, such as a pay zone. The spectral data was obtained by sampling the core at 15 cm intervals along the core of the vertical well referenced in FIG. 4. The five properties indicated are carbonate wavelength, carbonate, clay, water and TOC. As graphed, certain boundary areas can be discerned from the data. For example, depths of established pay zones, flow barriers and possible pay zones are identified by changes in the carbonate, clay, water and TOC. For example, high levels of clay may indicate a flow barrier.

The profiles obtained from the spectra enable an assessment of the location of pay zones. This could be established rapidly by the geologist based on core logging. As a result of this information, immediate adjustments may be made based on rapid determination of mineralogy. The spectral data may also facilitate a rapid determination of potential flow barriers. The spectral data may also be used for the identification of potential pay zones that may have been missed in traditional analysis. Identification of pay zones may increase the net to gross pay value of the well.

The spectral data may also provide a better definition of landing zones as a function of pay thickness and position of flow barriers.

This information may allow decisions to be made about the well being drilled, such as stopping the drilling of a well if it not likely to be profitable or making adjustments as to depths of further drilling.

With reference to FIG. 6, carbonate wavelength data based on the spectra are indicated for various depths for the two horizontal legs of the northern wells and the southern well of FIG. 4. Based on the carbonate wavelength, portions are indicated as being calcite (>2325), dolomite/calcite mixture (2324 to 2315) or dolomite (<2315). The carbonate mineralogy based on the carbonate wavelength can be generated as the well is being drilled with the results shown in real time, such as less than 1 minute or preferably less than 15 seconds from the spectra sample being taken of the drill cuttings. Alternatively, the carbonate data could be generated by sampling core samples in the lab after the well has been drilled. Such sampling may be used to generate a regional map view of mineralogical characteristics of cuttings from a suite of holes, and link to past production or other hole information, such as porosity, permeability, pay zone locations and hardness. The regional map view may be used to identify new well locations, identify locations to avoid where it may be depleted, identify potential missed opportunities with an existing play and potentially reduce risk for new well locations.

In the example of FIG. 6, the southern well displays a more consistent dolomitic composition throughout.

This information can be used to explain why the production differs between the northern and southern wells in this example. In this example, with reference to FIG. 4, the oil production for the southern well was higher than the northern well. As indicated in these example figures, the production differences are attributable to the dolomitic mineralogy of the productive well and consistent with the dolomitic mineralogy of the pay zones in the nearest vertical well.

The spectra data may also provide a real time decision tool to abandon a leg in the presence of unproductive mineralogy. For example, if a leg is being drilled and the carbonate wavelengths indicate properties similar to the less productive northern well, the well may be abandoned in favour of a more productive geology. This decision can be made as the results from the drilling cuttings are analyzed in minutes.

The spectra data may also allow comparative mineralogical data between wells to define variability across a field. This can be used to improve well placement. Without this information from the spectra data, mineralogical data may not otherwise be available at that scale.

The rapid mineralogical determinations from spectral data obtained from vertical core measurements and spectral data from cuttings in horizontal legs, enables a synergistic analysis that has not been achieved in a timely manner before. With the embodiment described, this analysis can be done quickly in the manner of minutes, or in some cases days. The data can be used to understand varying performance across fields and update and improve well placements.

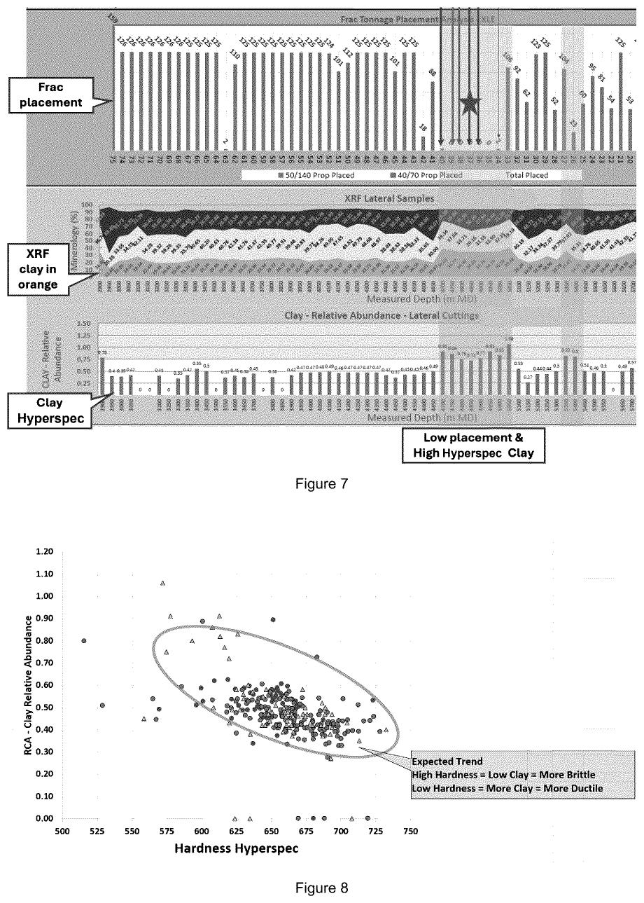

With reference to FIG. 7, elevated clay content estimated from hyperspectral data at a well site in accordance with an embodiment, correlates with clay content derived from X-ray fluorescence post drilling. Instances where clay content exceeds ˜30% correlated with poor frac placement. Clay content derived from hyperspectral data at the well can therefore be used to confirm steering within the target horizon to avoid and minimize high clay horizons and attempt to drill out of them faster. Access to live clay estimation immediately post drilling also serves in adjusting frac completion design.

FIG. 7 shows three rows representing data at corresponding depths for a well. The first row shows the frac placement. As indicated with the red star, a portion of low placement took place at a depth of between 4700 and 5050 metres. The second row shows post drilling mineralogy (blue: carbonates, yellow: silicates, orange: clays) derived from X-ray fluorescence from the lateral cuttings. This was calculated post drilling, typically weeks to months after the well was completed. Highest clay content is observed in the depth interval of poor frac placement (placement #34-40).

The bottom row represents the hyperspectral data obtained at the well site in accordance with an embodiment and would be available to the operator as the well was being drilled. The clay relative abundance obtained using the hyperspectral data shows higher levels of clay at the depth of 4700 to 5050 metres. This indication corresponds to the data seen in the post-drilling analysis. This confirms that the hyperspectral data can be used to infer depths of low placement, without requiring the post drilling analysis. Therefore, using the hyperspectral data at the well site, allows the operator to make decisions about steering and drill out of the clay areas faster.

FIG. 8 represents data relating hardness hyperspec derived from hyperspectral measurements done in accordance with an embodiment, with clay content measurements derived from X-ray fluorescence (typically done post-well completion) collected from lateral cuttings of a sample well. The horizontal axis indicates the hardness hyperspec derived from the hyperspectral measurements. The model predicting hardness from hyperspectral measurements was developed from impact tests on core conducted with an Equotip Bambino 2 and the measurements scaled to 0-1000. The vertical axis is the relative clay abundance (RCA). Cuttings from three laterals (green, orange, red symbols) were sampled showing the correlation between clay content and hardness hyperspec. High hardness results in more brittle rocks with enhanced fracturing potential.

With reference to FIG. 8, prediction of rock hardness from hyperspectral measurements at the well correlates well with clay content derived from X-ray fluorescence (typically post well completion). This is of particular value for frac completion design on pads/areas of higher clay risk. Hyperspec mineralogy and hardness can be related to the geological striplog well plots for drill steering. It can also be used for completions designs and post frac completion analysis, such as improve drilling and completions through mineralogical analysis.

The processing of the spectral data may include interpolation/resampling of the data to equal bandwidth, such as using a SPLINE method.

The processing of the spectral data may also include determining mineral composition and relative mineral abundance, and quantitative estimate of rock properties. A calibration may be required for a spectrometer, such as initially or periodically. This may consist of presenting a panel of know reflectance to the instrument and taking a spectrum from the panel. Subsequent cuttings spectra may be normalized to this measurement. This may be done automatically by the spectrometer instruments.

Determining mineral composition may include continuum removal (CR), detection of features and determining their position (wavelength) and strength (depth or peak), and assignment of feature position to mineral composition.

Quantitative estimation of rock properties may include the application of predictive models to the spectra data using calibration data. This may be used to quantify total organic carbon (TOC), mineralogy and rock hardness. This process of estimation may include spectral decomposition of reflectance spectrum using continuous wavelet analysis, feature selection from a correlation scalograms that indicates features in a suite of spectra that correlate to properties such as TOC of the material. Multivariable regression analysis may further establish the model to define combinations of features to predict the quantifiable properties. Once the model is developed spectra may be applied to the model to obtain the results.

In operation, the drilling of oil and gas reservoirs can be enhanced using the spectra measurements. The spectra measurements and analysis can assist with connecting rock composition to underlying geomechanical behaviour and to understand the scale at which the geomechanical properties vary. This may be helpful for predicting responses to hydraulic fracturing.

The system may be calibrated by taking spectra measurements of a core for which properties are already known. For example, a core where the TOC or hardness has already been analyzed at known intervals, may be used as data for a calibration model.

Various embodiments of the present disclosure having been thus described in detail by way of example, it will be apparent to those skilled in the art that variations and modifications may be made without departing from the disclosure. The disclosure includes all such variations and modifications as fall within the scope of the appended claims.

1. A method of controlling a drill for an oil or gas well comprising:
a. extracting drill cuttings from the drill;
b. obtaining a reflectance spectra of the drill cuttings while the drill is operating;
c. communicating the reflectance spectra over one or more communication networks to a remote repository;
d. processing the reflectance spectra at a remote server in communication with the remote repository, wherein processing the reflectance spectra comprises applying a model to obtain values for each of one or more properties of the drill cuttings;
e. communicating the values to a receiver proximate to the drill;
f. adjusting the drill based on the values.

2. The method of claim 1 wherein the reflectance spectra is obtained for a frequency range of 0.4-22 um.

3. The method of claim 1 where the remote server receives a notification from the remote repository when the reflectance spectra has been communicated to the remote repository.

4. The method of claim 1 further comprising storing the reflectance spectra at the remote repository, such as in a database.

5. The method of claim 1 wherein the processing comprises using a statistical relationship between one or more wavelet powers and the property of the reflectance spectra.

6. The method of claim 1 further comprises calibrating the obtaining of reflectance spectra using a sample of known properties and comparing the reflectance spectra with the spectra of the sample.

7. The method of claim 1 wherein adjusting the drill comprises one or more of stopping the drilling, changing a direction of the drill, changing a drilling mud being used, changing a depth of the well being drilled.

8. The method of claim 1 further comprising determining minerology for the well being drilled.

9. The method of claim 1 further comprising preparing a frac completion design using the values of the one or more properties of the drill cuttings.

10. A system for processing drill cuttings comprising:
a. a spectrometer for taking reflectance spectra of drill cuttings, the spectrometer in communication with a remote repository and configured to communicate the reflectance spectra to the remote repository;
b. a server in communication with a remote repository, configured to be notified of the communication of reflectance spectra from the spectrometer, and in response to the notification, processing the reflectance spectra by applying a model to obtain values for each of one or more properties of the drill cuttings;
c. a receiver proximate to the spectrometer in communication with the server with a display for displaying the values.

11. The system of claim 10 wherein the reflective spectra are in a frequency range of 0.4-22 um.

12. The system of claim 10 further comprising a storage device for storing the reflectance spectra at the remote repository.

13. The system of claim 10 wherein processing the reflectance spectra comprises using statistical relationship between a one or more wavelet powers and the property of the reflectance spectra.

14. The system of claim 10 wherein the spectrometer comprises a calibration mode, the calibration mode includes calibration of the spectra with a sample of known properties and comparing the reflectance spectra with the spectra of the sample.

15. The system of claim 10 further comprising displaying minerology of the drill cuttings.
